(19) United States
(12) Patent Application Publication
(10) Pub. No.: US 2024/0238533 A1
(43) Pub. Date: Jul. 18, 2024

(54) SAFETY NEEDLE ASSEMBLY

(71) Applicant: Terumo Europe NV, Leuven (BE)

(72) Inventors: Ludo Willy P DANIELS, Tongeren (BE); Pieter Nico Jan CASTELEYN, Herent (BE); Hans VALKENAERS, Booischot (BE); Christian Roland PP FRIPON, Haasrode (BE)

(21) Appl. No.: 18/561,871

(22) PCT Filed: May 18, 2022
(86) PCT No.: PCT/EP2022/063474

§ 371 Date: Nov. 17, 2023

(30) Foreign Application Priority Data
May 18, 2021 (WO) PCT/EP2021/063205

(51) Int. Cl. A61M 5/32

(52) U.S. Cl. CPC A61M 5/322; A61M 5/3216

(57) ABSTRACT

Some embodiments are directed toward a safety needle assembly including a hub having a proximal end for connecting with a syringe extending in a proximal area of the hub; a needle longitudinally extending from a distal end of the hub in a distal area of the hub; and a sheath pivotally connected to the hub. The sheath can have two side walls connected by a back wall defining a receiving space therebetween for receiving the needle; wherein the sheath is adjustable between a starting position in which the sheath is substantially positioned in the distal area of the hub, between a use position in which the sheath is substantially positioned in the proximal area of the hub, and between a locked position in which the sheath is locked to secure the needle into the receiving space.

### CROSS REFERENCE TO RELATED APPLICATIONS

This application is a national phase filing under 35 C.F.R. § 371 of and claims priority to PCT Patent Application No. PCT/EP2022/063474, filed on May 18, 2022, which claims the priority benefit under 35 U.S.C. § 119 of PCT Patent Application No. PCT/EP2021/063205, filed on May 18, 2021, the contents of which are hereby incorporated in their entireties by reference.

### BACKGROUND

The invention relates to a safety needle assembly. Safety needle assemblies are widely known and used. Typically, a safety needle assembly comprises a hub having a proximal end for connecting with a syringe. Further, there is a needle longitudinally extending from a distal end of the hub. When the hub is connected to the syringe, a fluid connection between the needle and the syringe is established such that fluid from the syringe can be injected via the needle. Usually, the safety needle assembly comprises a sheath that is pivotally connected to the hub. The sheath typically has two side walls connected by a back wall defining a receiving space therebetween for receiving the needle. The sheath is pivotable between a starting position in which the sheath is free from the needle, between a use position in which the sheath is pivoted away from the needle and the needle is exposed, and between a locked position in which the sheath is locked to secure the needle into the receiving space. In the locked position, the sheath protects the needle, after its use, against unintended contact, contamination, needlestick injury etc. In the locked position, the needle is received in the receiving space of the sheath to be enclosed by the sheath, and as such, to cover the needle.

### SUMMARY

Prior to the use, the user, e.g. a paramedical person, partially opens the blister packaging to make the hub free, and then holds the assembly at the packaging to connect the hub to the syringe to avoid contamination or a possible sterility breach of the needle assembly. Then, the user further removes the packaging to make the safety needle assembly free. The user then pivots the sheath from its starting position towards the use position and removes the protector from the needle to expose the needle such that the needle can be used. After the injection, the needle is retracted and the sheath is pivoted from the use position towards the locking position to lock the needle to the sheath. The handling of the blister packaging is to be done carefully to avoid contamination of the sterility of the safety needle assembly.

In the starting position of the sheath, the safety needle assembly is being packaged and transported. Typically, the safety needle assembly with the sheath in the starting position is packaged in a blister packaging. In the starting position, the sheath is free from the needle, the sheath is not covering the needle in the starting position, so, to protect the needle, an additional protector is provided. The safety needle assembly is packaged sterile. The safety needle assembly is relatively bulky due to the fact that the sheath is in a specific angle in the starting position, resulting in a rather large footprint. The specific angle of the sheath in the starting position is usually relatively large with respect to the needle, typically about 30 degrees. The specific angles of the starting position, use position and locked position are sometimes fixed at the hinge or via snaps. The relatively large angle of the sheath in the starting position is due to the momentum, and thus angle, needed to lock the sheath in the locked position, as well as for a firm positioning of sheath in the starting position. Since the starting position is also the position in which the sheath is positioned during packaging and transporting of the needle assembly, this position is preferably held relatively firmly to avoid the sheath from unintentionally further closing or opening. So, in that position some holding elements are provided to hold the sheath in starting position, and/or in use position. The momentum required to lock the sheath is determined by the locking connection, often a hooked connection. When pivoting the sheath from the use position via the starting position towards the locked position, the starting position needs to be passed, and the sheath needs to be pushed through the starting position. From there on, the angular displacement needs to be sufficiently large to be able to hook the sheath behind the needle or behind the hub, or otherwise and thus to at least partially cover the needle to protect the needle and/or a user from needlestick injury, contamination etc.

At least one drawback of the present safety needle assemblies is that these are relatively bulky and have a relatively large footprint, which is disadvantageous for transport and packaging. Also, due to the large footprint packaging of individual needle assemblies is done in a blister packaging, which may make them difficult to handle.

There is a need for a safety needle assembly that resolves at least one of the abovementioned drawbacks.

Thereto, the invention provides for a safety needle assembly according to claim 1 or to claim 2.

By providing a safety needle assembly wherein the starting position of the sheath is longitudinally extending from the hub, the sheath is positioned approximately parallel to the needle, and thus may enclose the needle already wholly or partially in the starting position. In the starting position, the sheath is positioned substantially at the same side of the hub as the needle is extending, thus the sheath is positioned in the distal area with respect to the hub. In the use position, the sheath is pivoted away from the needle towards a position substantially extending at the other side of the hub, in the proximal are of the hub, where, in use, the syringe is connected. A safety needle for which the user actively needs to move the sheath from the starting position to the use position and/or the locked position, is known as an active safety needle. As such, the user activates the sheath by pivoting the sheath away from the starting position and allowing the needle to be exposed.

By providing the sheath longitudinally extending from the hub in the starting position, the footprint of the safety needle assembly in the starting position, thus for packaging purposes, becomes much less, allowing different types of packaging and/or more efficient packaging and/or transport and/or storage. For example, which such a reduced footprint, a hard case packaging may be considered, allowing automated handling of the packed safety needle assembly. Whereas, automated handling of blister packed assemblies is practically not possible, resulting that the blister packed assemblies need to be collected manually in a box for transport and storage. This limits the speed of assembly lines. By providing the sheath in a longitudinally extended position from the hub in starting position, a more compact arrangement of the safety needle assembly can be obtained, allowing a hard case packaging.

Such hard case packaging can be handled automated allowing the speed of the assembly lines to increase, thus allowing a more efficient production, packaging, transport and/or storage of the safety needle assembly. Also, by using a hard cap, the way of working for preparing the needle assembly to use remains unchanged. This is advantageous for the user as he can follow the procedure he is used to. When removing the cap of the hard case, the hub of the needle assembly becomes exposed and can be connected to the syringe without compromising the sterility and avoiding possible contamination. When connected to the syringe, the hard case can be removed from the needle assembly and the sheath can be pivoted to the use position to make the needle with syringe ready for use.

Further, when the sheath is in a longitudinally extending position in the starting position, the receiving space formed by the sheath may enclose the needle. The sheath may be in a position approximately parallel to the needle, so the needle can be received in the receiving space relatively easy. Thus, the sheath may form a protection for the needle in the starting position. Thereby, a needle protector, necessary in the prior art safety needle devices to protect the needle when the sheath is in the starting position, may then be thought to omit.

Additionally and/or alternatively, the hub can be provided with at least one first cooperating element and the sheath can be provided with at least one second cooperating element, wherein, in the locked position of the sheath, the first and the second cooperating elements are engaged to each other for locking the sheath. Advantageously, by providing the locking elements on the hub and the sheath respectively, a more firm locking can be obtained in the locked position of the sheath to provide for protection of the needle after use. Contrary to prior art needle assemblies, in which the sheath may lock around the needle itself, providing the locking elements on the sheath and the hub, may allow for a more stable and reliable securing of the sheath in the locked position.

Advantageously, in the starting position, the first and second cooperating elements are in contact with each other for defining the starting position of the sheath. The second cooperating element of the sheath may then rest on the first cooperating element of the hub, thereby a restraint or a stop may be defined preventing the second cooperating element of the sheath to pass the first cooperating element of the hub. As such, the starting position of the sheath can be defined by the cooperating elements themselves, and a reliable positioning of the sheath in the starting position can be obtained. Additionally, when pivoting the sheath from the use position to the locked position, the locking elements pass the starting position, and will have to be pushed through the contact of the starting position, thereby giving a tactile indication to the user that the locked position is reached.

Preferably, the at least one first cooperating element extends longitudinally from the distal end of the hub. As such, the at least one first cooperating element may extend longitudinally from the hub in a direction approximately parallel to the needle along the needle. The at least one first cooperating element can be a hook, or a finger, or any other protruding element that can cooperate with another element to secure the sheath in the locked position. One or more first cooperating elements can be provided that can cooperate with one or more associated second cooperating elements of the sheath.

The at least one second cooperating element may be arranged in the receiving space of the sheath, at a position corresponding with their associated first cooperating element, such that, in the locked position the associated first and second cooperating elements engage to secure the sheath. Advantageously, the at least one second cooperating element may protrude from the back wall of the sheath into the receiving space. For example, a hook or a finger may protrude from the back wall, or from the side walls, to engage with the associated first cooperating element, e.g. a longitudinally extending finger or hook. Alternatively, or additionally, the at least one second cooperating element may be provided as an opening in the sheath. The associated first cooperating element may then engage through this opening to secure the sheath in the locked position. In the starting position, the first cooperating element may rest on or in the opening of the sheath to define the starting position. In the starting position, the first and second cooperating elements engage with each other defining the starting position, for example by abutting to each other. When the second cooperating element rests on the first cooperating element in the starting position, i.e. having contact with each other, this defines the starting position in a stable manner, without having the possibility of accidental engaging to the locked position.

For example, at least one of the first and second cooperating elements is provided as a snap fit element that engages in a snap-fit connection in the locked position of the sheath. For example, the first cooperating element may be a snap finger that snaps in the opening as second cooperating element to secure the sheath in the locked position. Additionally, or, alternatively, the second cooperating element may be a snap hook that snaps behind a longitudinally protruding finger as first cooperating element, or may snap behind some sideward protrusions on the hub. Of course, a combination of various embodiments of cooperating elements may be possible, e.g. there may be provided an opening in the back wall of the sheath that engages with a snap hook longitudinally protruding from the hub. This may be combined, or may be replaced, with snap fingers on side walls of the sheath that engage with protrusions extending side wards from the hub. Many combinations are possible.

Advantageously, the first and second cooperating elements may each be provided with inclined contact surfaces that are configured for contacting each other in the starting position of the sheath, the contact surfaces having the same inclination angle. By providing associated contact surfaces to the associated cooperating elements, that, in the starting position, resting onto each other, or abutting to each other, a reliable positioning of the cooperating elements in the starting position can be obtained. Due to the inclination of the surfaces, when pushing the sheath further through towards the locked position, the surfaces may slide along each other to allow locking of the cooperating elements. Advantageously, the first and second cooperating elements further are provided with further contact surfaces, at a different angle than the inclined contact surfaces, wherein the further contact surfaces are configured for contacting each other in the locked position of the sheath. The inclined contact surfaces and the further contact surface may then provide for a hook shape optimally providing for the snap-fit connection. In the starting position of the sheath, the first and second contact surfaces of the first and second cooperating elements may abut each other. Advantageously, the inclination of the contact surfaces is such that in the starting position, a stable arrangement of the sheath with respect to the hub is obtained, such that, e.g. during transport there is no accidental engaging of the cooperating elements. Also, the inclination of the contact surfaces is preferably such that, for movement towards the locked position, the user may have to apply some force, to push the sheath into the locked position, but this force should be acceptable for a user. The force which is necessary for bringing the sheath into the locked position should be significantly larger, e.g. 5 until 10 times larger, than forces necessary to move the sheath otherwise. The force for bringing the sheath in the locked position may e.g. be within the range of 1-10 N, preferably 2 N to 7 N, more preferably may be 3 N.

Advantageously, the first cooperating element that is mounted to the hub, protrudes in a direction away from the hub parallel to the cannula extending from the hub. A distal surface, being the first contact surface, of the first cooperating element is advantageously inclined, or chamfered, preferably with an angle of about 45 degrees with respect to the longitudinal direction of the first cooperating element. The second cooperating element advantageously is a hook, comprising a longitudinal body and a hooked end, protruding from the sheath, in particular from the back wall of the sheath, into the receiving space, in a direction approximately parallel to the side walls. An outer end of the second cooperating element is preferably provided as a hooked end, angled with respect to the longitudinal body at about 90 degrees. An end surface, being the second contact surface, of the hooked end is inclined, or chamfered, with respect to a longitudinal direction of the hooked end at about 45 degrees. In any event is the angle of the first and second contact surface the same, such that the contact surfaces in the starting position abut against each other defining the starting position. In the locked position, the hooked end of the second cooperating element engages with the first cooperating element, in particular an upper surface of the hooked end, engages with a bottom surface of the longitudinal first cooperating element, such that the hooked end is snapped behind the first cooperating element. A disengaging force for disengaging the hooked end from the first cooperating element is than larger than a force induced by e.g. accidental operations or transport or further handling, and also provides sufficient resistance for a user to disengage. The outer end of the first cooperating element is then in fact captured in an inner corner of the second cooperating element formed by the hooked end and the longitudinal body thereof.

Advantageously, a length of the first cooperating element is shorter than a length of the hub. Since the second cooperating element is configured to engage with the first cooperating element, the second cooperating element is positioned relatively shortly to the hub as well, and thus, relatively shortly to a rotation axis of the sheath with respect to the hub. This gives a relatively short moment arm, allowing a sufficient stable configuration of the first and second cooperating elements in the starting position, in that no accidental engagement due to e.g. handling or transport is possible. Also, the short moment arm provides for the user to adapt sufficient force to push the sheath to the locked position, in that the user really feels a resistance he has to push through, thus giving the user confidence that the sheath is in the locked position. The user may be provided with feedback to know that the sheath is in the locked position by this resistance, and may also be provided with feedback by a sound generated during engagement of the first cooperating element and the second cooperating element. Further, in case the sheath material is arranged as a transparent material, the user may more certainly assume that the sheath is in the locked position by visually inspecting the needle assembly.

Preferably, the angular displacement of the sheath between the starting position and the locked position of the sheath is less than 20 degrees, preferably less than 15 degrees, more preferably less than 8 degrees. In an advantageous embodiment, the angular displacement between the starting position and the locked position of the sheath is about 6 degrees. By providing such a limited angular displacement, the footprint of the needle assembly in particular in the starting position, as well as in the locked position can be relatively small.

Advantageously, the angular displacement of the sheath between the starting position and the use position is more than 90 degrees, preferably more than 110 degrees, more preferably more than 120 degrees. By having a large angular displacement between the starting position and the use position, the sheath is away from the needle and the needle becomes fully exposed for injection. Also, by providing such a large angular displacement, the user needs to actively pivot the sheath, and the needle cannot be exposed accidentally.

Further, the safety needle assembly may comprise a protector removable engageable to the distal end of the hub for protecting the needle when the sheath is in the starting position. Although in some embodiments, the protector may be omitted, because it is thought that the sheath in the starting position provides sufficient protection for the needle when the needle is at least partially received in the receiving space of the sheath. A protector may be provided to provide for additional protection. Also, when a user opens the sheath by pivoting it away from the starting position to the use position, the protector remains around the needle to protect the user from accidental needle injury. Furthermore, the protector may also prevent activation of the sheath during packaging, transport and/or handling. The contact of the first and second cooperating elements defining the starting position may be undone by forces exerted on the assembly during transport and/or handling, thereby undoing the protection of the needle by the sheath, or, accidentally activating the locked position. Providing a protector around the needle, engaged with the hub, may prevent such an accidental activation of the sheath. The protector may further be provided, at an inner side thereof, with longitudinally extending ribs that tightly engage with the hub. As such, the contact area between the protector and the hub may be reduced, while a firm connection with the hub is obtained and, also, a relatively easy removal. Preferably, the tight fitting of the protector to the hub may be obtained by the ribs protruding somewhat further outwardly than the outer diameter of the hub, such that a clamping engagement can be obtained. Of course, instead of ribs in the protector, the hub can be provided with outwardly extending longitudinal ribs. These ribs preferably have an outer diameter somewhat larger than the inner diameter of the protector to provide for the tight fit engagement.

Additionally, the sheath may comprise bridge elements arranged in the receiving space which bridge elements support on the protector in the starting position of the sheath. By providing these bridge elements, the sheath can be held in the starting position, not only by the first and second cooperating elements resting onto each other, but also by the bridge elements abutting the protector. This provides additional safety for the assembly during packaging, transport and/or handling when the assembly may be subject to forces. The bridge elements may prevent accidental activation of the sheath in the starting position.

The sheath may further be provided with an additional cooperating element for engaging with a distal end of the needle in the locked position of the sheath. In particular with relatively long needles, an additional connection point is foreseen to hold the relatively long needle securely in the receiving space. The additional cooperating element can be provided as a snap hook extending in the receiving space behind which the distal end of the needle may snap fit when the sheath is secured in the locked position.

The sheath may comprise a recess region to engage with a finger, e.g. an index finger or a thumb, of a user, such that the user can pivot the sheath between the starting position, the use position and/or the locked position. By providing such a recess region, the user can more easily manipulate the sheath. Alternatively, pivoting of the sheath, in particular opening or activation of the sheath, can be done by pushing a tip, distal end, of the sheath on a hard surface, e.g. a table top. The recess region may for example be provided in the back wall of the sheath, such that the sheath can be activated by manipulation by a single finger only. Alternatively, the recess region, or recess regions, may be provided in one or in the two side walls of the sheath.

Further aspects of the disclosure relate to a set of a safety needle assembly and a hard case in which the set is stored, as well as to the hard case in which the safety needle assembly, with the sheath in the starting position, can be inserted, as well as to a kit of a hard case and a safety needle assembly.

Further advantageous embodiments are represented in the subclaims.

### BRIEF DESCRIPTION OF THE DRAWINGS

These and other aspects will further be elucidated with reference to the drawing comprising figures of exemplary embodiments. In the drawing shows:

FIG. 1 an exploded view of a safety needle assembly;

FIG. 2a a perspective view of the safety needle assembly in the starting position;

FIG. 2b a perspective view of the safety needle assembly in the use position with the protector covering the needle;

FIG. 2c a perspective view of the safety needle assembly in the use position without the protector;

FIG. 2d a perspective view of the safety needle assembly in the locked position;

FIG. 3a a detail of the first and second cooperating elements in the starting position;

FIG. 3b a detail of the sheath in the use position;

FIG. 3c a detail of the first and second cooperating elements in the locked position;

FIG. 4 a side view of the assembly with the sheath in the different positions indicating the angular displacement of the sheath;

FIG. 5a a longitudinal cross-section of the safety needle assembly in the starting position with the sheath,

FIG. 5b a detail of a bridge element of the sheath supporting on the protector;

FIG. 5c a detail of the first and second cooperating elements in the starting position;

FIG. 6 a side view with transparent sheath in the locked position with a long needle;

FIG. 7 a longitudinal cross-section of the sheath in the locked position with a long needle;

FIG. 8a a perspective view of the assembly in a hard shell case;

FIG. 8b a perspective view of the case packaging closed with a cap;

FIG. 9a a longitudinal cross-section of an alternative embodiment of first and second cooperating elements, with the sheath in the starting position;

FIG. 9b a longitudinal cross-section of the embodiment of FIG. 9a with the sheath in the locked position;

FIG. 10a longitudinal cross-section of an alternative embodiment of the first and second cooperating elements with the sheath in the starting position;

FIG. 10b a transverse cross-section of the embodiment of FIG. 10a with the sheath in the starting position;

FIG. 10c a transverse cross-section of the embodiment of FIG. 10a with the sheath in the locked position;

FIG. 11a longitudinal cross-section of an alternative embodiment of the first and second cooperating elements with the sheath in the starting position;

FIG. 11b a transverse cross-section of the embodiment of FIG. 11a with the sheath in the starting position;

FIG. 11c a transverse cross-section of the embodiment of FIG. 11a with the sheath in the locked position;

FIG. 12a a perspective view of an alternative embodiment of the assembly with a longitudinal cross-section of the sheath and hub, with the sheath in the starting position;

FIG. 12b a perspective view of the embodiment of FIG. 12a with the sheath in the use position:

FIG. 12c a perspective view of the embodiment of FIG. 12a with a longitudinal cross-section of the sheath and hub, with the sheath in the locked position:

FIG. 12d a transverse cross-section of the embodiment of FIG. 12a with the first and second cooperating elements in the locked position:

FIG. 13a a detail of the hub of the needle assembly:

FIG. 13b a front view of the hub of FIG. 13a:

FIG. 14 a schematic view of a hub of the needle assembly:

FIG. 15 a schematic view of the hub of FIG. 14;

FIG. 16 a cross-section of the hub of FIG. 14.

### DETAILED DESCRIPTION OF EMBODIMENTS

It is to be noted that the figures are given by way of exemplary examples and are not limiting to the disclosure. The drawings may not be to scale. Corresponding elements are designated with corresponding reference signs.

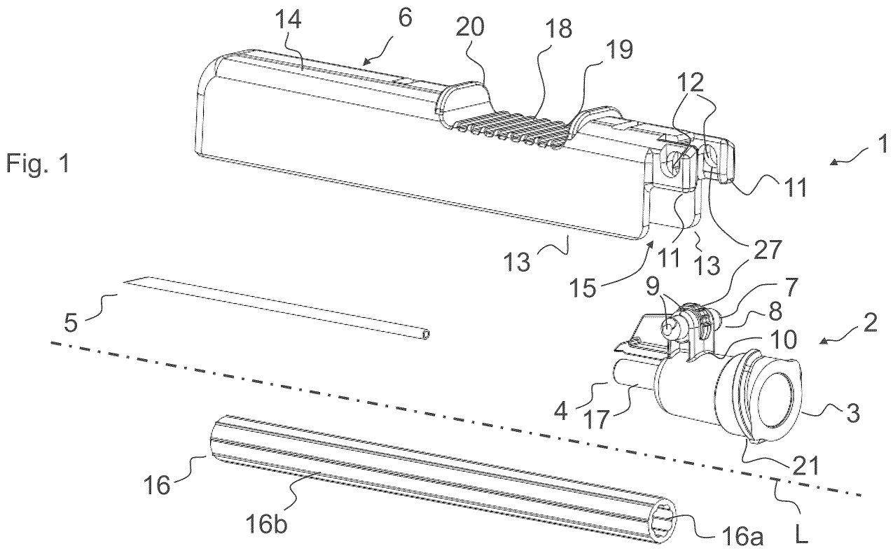

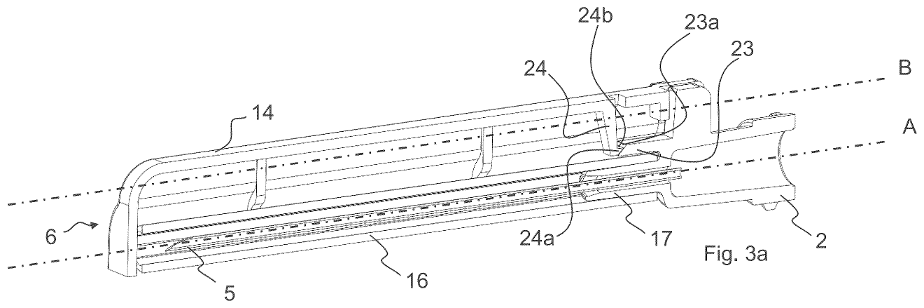

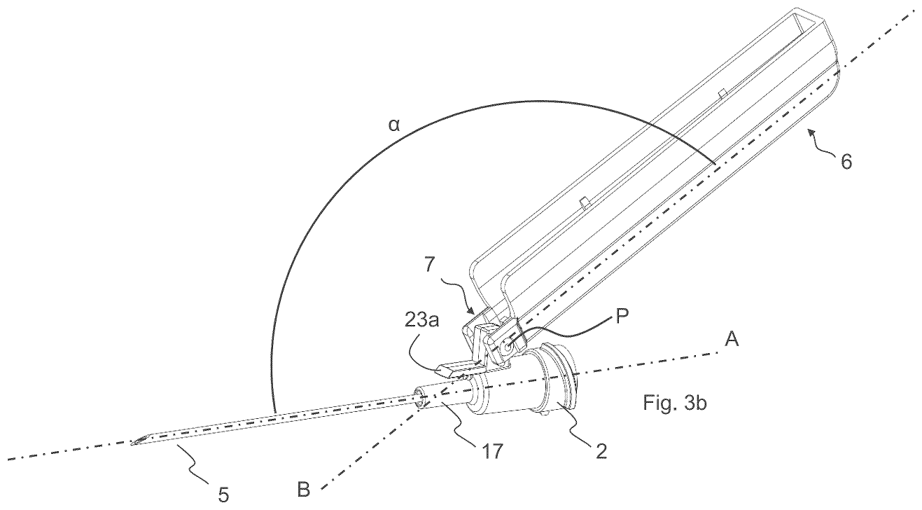

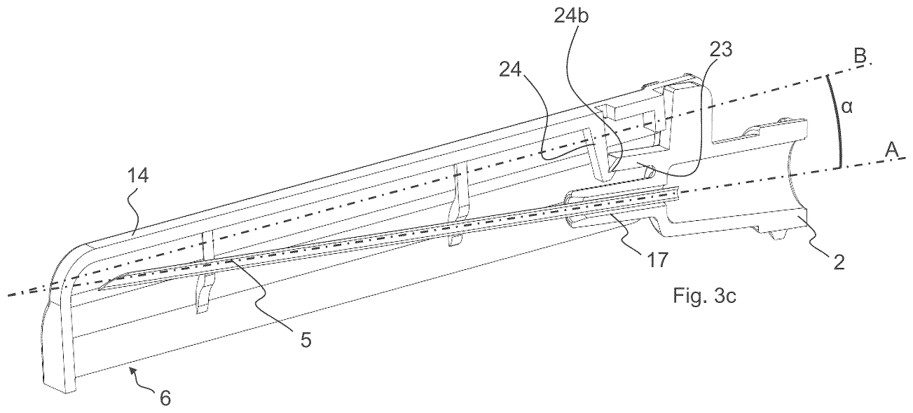

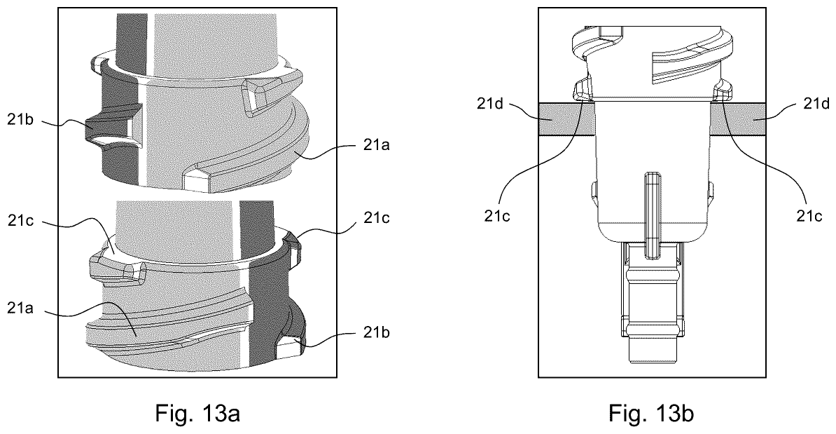

FIG. 1 shows an exploded view of a safety needle assembly 1. The safety needle assembly 1 comprises a hub 2 having a proximal end 3 for connecting with a syringe in a proximal area of the hub 2. The syringe is not shown in the figure. The hub 2 further has a distal end 4 to which a needle 5 can be connected. The needle 5 longitudinally extends from the distal end 4 from the hub 2 along a longitudinal direction L. The distal end 4 of the hub 2, can, to that end, be provided with a protrusion 17 in which the needle 5 can be received. So, the side of the hub 2 where the syringe is to be connected can be considered as the proximal side or the proximal area of the hub, and the side of the hub 2 where the needle 5 is connected can be considered as the distal side or the distal area of the hub. The hub 2 may comprise an inner surface tapering towards the distal side of the hub 2, e.g. in accordance with the appropriate ISO norm for a needle assembly (e.g. ISO 80369-7:2021 relating to the proximal side of the hub 2) having length of 7.5 mm, see FIG. 3a and further. For connecting a syringe to the proximal end 3 of the hub 2, the proximal end 3 may for example be provided with a thread 21. The connection with the syringe may be a screw connection, but can be a luer lock connection or a slip tip connection as well. The thread 21 may comprise only thread segments 21a, 21b that remain within predefined sections along a circumference surface of the hub 2. The predefined sections may e.g. comprise two hemi-cylinder-like outer surfaces, see FIG. 13a. As such, in case the hub 2 is manufactured using a molding processing technique comprising a two-piece mold, the thread can be produced without gaps within the thread segments 21a, 21b. Further, an end of the thread may comprise a flat surface 21c, parallel to a cross-section of the hub 2, such that the hub 2 can be moved by exerting a force orthogonally onto the surface 21c, e.g. during a manufacturing feeding process. This is illustrated in FIG. 13b, wherein areas :2-1d marked in grey represent the locations where a means could exert a force on the hub 2 for feeding the hub 2 during a manufacturing process. The thread 21 may be arranged such that a maximal contact surface with the syringe is provided in order to prevent the syringe to become loose. In relation to a molding manufacturing process, the sheath 6 may be provided with appropriate molding holes to prevent movable parts in the mold.

Further, the safety needle assembly 1 is provided with a sheath 6 that is pivotally connected to the hub 2. The pivot connection 7, e.g. a hinge, is here embodied as an axle 8 transverse oriented with respect to the longitudinal direction L. The axle 8 has exposed ends 9 extending from a rib 10 mounted to the hub 2. The sheath 6 is provided with two longitudinally oriented flanges 11 each having an opening or hole 12 therein, which openings 12 engage around exposed ends axle ends 9 to form the pivot connection 7. Other embodiments of a pivot connection are possible. An exposed end 9 may be configured as a transverse chamfer, which can simplify mounting of the sheath 6. A corresponding opening 12 of sheath 6 may correspondingly be configured as a transverse chamfer.

The sheath 6 has two side walls 13 extending over at least a part of a length of the sheath 6. The side walls 13 are connected by a back wall 14. The side walls 13 and the back wall 14 define a receiving space 15 therebetween for receiving the needle 5.

Optionally, the safety needle assembly 1 can be provided with a protector 16. The protector 16 is typically embodied as a longitudinally extending sleeve that can be engaged over the needle 5, and at an end there of can be engaged to the distal end 4 of the hub 2, e.g. by providing an inner surface of the protector 16 with a rippled cross-section. The protrusion 17 of the distal end 4 of the hub 2 can, to that end, be provided with engagement elements 17a such as ribs or grooves to clampingly receive the protector 16. The ribs or grooves 17a can be arranged transversally and/or longitudinally. By as such reducing the contact area between the protector 16 and the hub 2, a force exerted on the protector 16, e.g. for pulling off the protector 16, can be better controlled by a user of the safety needle assembly 1. An outer surface of the protector 16 may also be provided with ribs, grooves or the like, for altering the contact area between the protector 16 and another part of the hub 2 or of the sheath 6. The distal end 4 of the hub 2 may also comprise a stopper surface 34 for more easily mounting the protector 16 to the hub 2, such that an end of the protector 16 can abut against the stopper surface 34, providing extra stability to the safety needle assembly 1. The stopper surface 34 may e.g. be configured as a surface arranged along part of an outer surface of the protrusion 17, e.g. extending from the outer surface of the protrusion 17 to a part of the hub 2, e.g. cooperating element 23. The protector 16 is removably connected to the hub 2. The protector is here provided with longitudinally extending ribs 16a at an inner side of the protector. The longitudinally extending ribs 16a provide for contact to the protrusion 17 in an efficient manner. By providing the ribs, less contact area is possible, while sufficient clamping can nevertheless be obtained. Preferably, the ribs 16a have height such that the outer diameter of the protrusion 17 is somewhat larger than the inner diameter of the protector 16 formed by the ribs 16a, as to ensure a reliable clamping, while also allowing a relative easy removal. The ribs 16a may be arranged as to form a flower-structure inside of the protector 16, but other arrangements may be possible. Alternatively, ribs may be provided onto the protrusion 17 engaging to a smooth inner surface of the protector 16. Further, in this example, the protector 16 is also, at its outer surface, provided with longitudinally extending ribs 16b. These longitudinally extending ribs 16b may allow placing of the protector 16, e.g. after removal, on a hard surface such as a table top, without rolling over. For a physicist or user, working with the needle assembly 1 it is important to keep all parts together, and not to be searching after parts that have been rolling away or have been rolling from the table. The ribs 16b may prevent such rolling away. Of course, other configurations may be thought of that may prevent rolling away, such as wings or other protrusions. The protector 16 prevents wrongful activation of the locked position during movement, e.g. during transport or during attachment of the syringe to the needle assembly 1. In use, the protector 16 is preferably removed from the needle assembly 1 in order not to obstruct the switching between positions of the needle assembly 1. In order to draw the attention of the user to the protector 16, the protector 16 may comprise an outer surface arranged in a look, e.g. by a different color, material or texture, which is different from the other parts of the needle assembly. As such, the differing look of the protector 16 may be a visual cue for the user to remember to remove the protector 16 before use. Alternatively, the protector 16 may be arranged such that it does not need to be removed during use, but can still provide its blocking functionality in order to prevent the needle assembly to reach the locked position by accident, e.g. by choosing an appropriate material or material thickness.

The sheath 6 is adjustable between a starting position in which the sheath 6 is substantially positioned in the distal area of the hub 2, between a use position in which the sheath is substantially positioned in the proximal area of the hub 2, and between a locked position in which the sheath 6 is locked to secure the needle into the receiving space 15 of the sheath 6. In FIGS. 1 and 13, it can be seen that on the pivot connection 7 an adjustment structure 27 is provided. The adjustment structure 27 here comprises a pattern of recesses and ribs. In the recesses an associated element, e.g. a rib, of the sheath 6 fits such that the sheath 6 can be held in angular position in the said recess. By pushing the sheath 6 further over a rib to the next recess, the angular position of the sheath 6 can be adjusted, as well as that the sheath 6 can remain in the next angular position. By providing such a pattern of recesses and ribs, the angular position of the sheath 6 can be adjusted while providing to the user a tactile feedback every time the sheath 6 is pushed over such a rib of the adjustment structure 27. Further, the sheath 6 can be held in many angular positions, in addition to the starting position, the use position and the locked position, in a stable and reliable manner.

In the example of FIG. 1, the back wall 14 of the sheath 6 is provided with a region 18 to engage with a finger of a user, e.g. an index finger. The region 18 can be a roughened area, e.g. provided with ripples, ribs or other roughening elements 19, or, as in the embodiment of FIG. 1, can be provided as a recess region 20 in the back wall 14 of the sheath. The region 18 may be configured as an anti-slip material. Alternatively, the region 18 may be provided on the side walls 13 of the sheath 6. A combination of a recess 20 with roughening elements 19, as in FIG. 1, is also possible. It is also possible to arrange multiple regions 18 along an outer surface of the sheath 6 which may or may not comprise a roughened area/roughening elements 19.

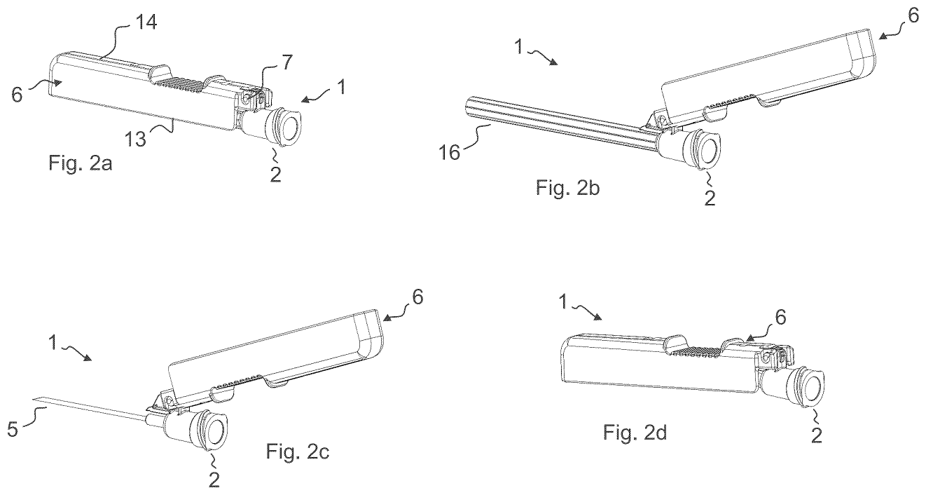

FIGS. 2a-2d show a sequence of adjusting the sheath 6 between the starting position and the locked position. FIG. 2a shows the sheath 6 connected to the hub 2, wherein the sheath 6 is positioned in the starting position. In the starting position, the sheath 6 is positioned in the distal area of the hub 2, i.e. the area of the hub 2 where also the needle 5 is arranged. In this embodiment of the sheath 6, with the two side walls 13 and the back wall 14 forming a U-shaped receiving space 15, the needle can be received in the sheath 6. The sheath 6 also covers the needle 5 in the starting position. This is contrary to the prior art safety needle assemblies in which the sheath in the starting position is entirely free of the needle. Here, the shape of the side walls 13 is such as to enclose the needle 5, but with an alternative shape, the side walls 13 may partially expose the needle 5. In the starting position, the sheath 6 is longitudinally extending from the hub 2. The sheath 6 is thus preferably extending in the same direction L as the needle 5. In some embodiments, it may be said that the sheath 6 is oriented parallel to the needle 5. By providing the sheath 6 in the starting position in a position longitudinally extending from the hub 2, the foot print of the safety needle assembly becomes rather limited, allowing the safety needle assembly 1 to be packaged in a packaging have a limited volume. This provides for less bulky packaging, reducing transport and storage costs. In a beneficial embodiment, the packaging can be a hard case packaging, allowing automated handling of the packaging during manufacturing and assembly, thereby reducing manufacturing costs.

After removing the safety needle assembly 1 from its packaging, typically the syringe is connected to the hub then, the user may open the sheath 6 and may pivot the sheath 6 to the use position, as shown in FIG. 2b and FIG. 2c. The safety needle assembly 1 is typically an active safety needle assembly 1 in which the user needs to actively pivot away the sheath 6 from its starting position to allow the needle 5 to be exposed. In the use position, or the working position, the sheath 6 is pivoted away from the needle 5 and is positioned substantially in the proximal area of the hub 2. When pivoting the sheath 6 to the use position, away from the needle 5, the needle 5 becomes exposed and can be used for injection. If the safety needle assembly 1 is provided with a protector 16 engaged around the needle 5, the protector 16 can be removed prior to the use of the needle 5.

After use of the needle 5, after injection, the needle 5 is to be protected again e.g. to prevent accidental contact or other misuse. To protect the needle 5 after use, the sheath 6 is pivoted towards the needle 5 again to a locked position as shown in FIG. 2d. In the locked position, the needle 5 is secured into the sheath 6. The sheath 6 can be connected to the hub 2 in a securing manner and/or can be connected to the needle 5 in a securing manner. For example, a second hook extending from the back wall 14 or from a side wall 13 of the sheath 6, into the receiving space 15 of the sheath 6, may snap around the needle in the locked position. The position and shape of the second hook within the sheath 6 may depend on the needle diameter and the needle bevel. In addition, the second hook may be arranged such that in the starting position and/or the use position, the second hook does not touch the needle. The second hook may also be arranged such that in any position, the needle does not make contact with the sheath 6. As can be seen in FIG. 2d, in the locked position the sheath 6 is pivoted further towards the needle 5 than in the starting position of the sheath 6. So, a user wishing to secure the needle 5 to the sheath 6, may pivot the sheath 6 from the working position, as shown in FIG. 2c through the starting position towards the locked position as shown in FIG. 2d.

By pivoting the sheath 6 from the starting position to the use position and to the locked position, an angular position of the sheath 6 with respect to the needle 5 is adjusted. The angular position of the sheath 6 with respect to the needle 5 can be indicated by the angle alpha, as shown in FIGS. 3a-3c. The angle alpha can be considered as the angle between a longitudinal axis A of the needle 5 and a longitudinal axis B of the sheath 6, typically the longitudinal axis B of the sheath 6 is through a pivot axis P of the pivot connection 7.

As can be seen in FIG. 3a, the longitudinal axis B is parallel or approximately parallel to the longitudinal axis A. Typically, the angle alpha is in the starting position between about 0 to about 20 degrees, allowing for the small foot print of the assembly 1 in the starting position. When pivoting the sheath 6 to the use position, as shown in FIG. 3b, the angle alpha between the longitudinal axis B and the longitudinal axis A becomes larger, typically around 160 degrees, preferably between about 120 degrees and about 170 degrees. With such a large angle alpha, the needle 5 becomes fully exposed for injection.

When pivoting the sheath 6 from the use position of FIG. 3b to the locked position of FIG. 3c, the angle alpha between the longitudinal axis B and the longitudinal axis A becomes zero to negative, as the locked position is further pivoted towards the needle 5 than the starting position. Typically, the angle alpha in the locked position is between about zero degrees and about minus 15 degrees, preferably between about zero degrees and about minus 10 degrees, more preferably, the angle alpha in the locked position is about minus 6 degrees. It is understood that, when the angle alpha in the locked position is smaller than the angle alpha in the starting position, the angle alpha in the locked position cannot be equal to the angle alpha in the starting position. For example, if the angle alpha in the starting position is zero degrees, then, the angle alpha in the locked position is smaller than zero degrees.

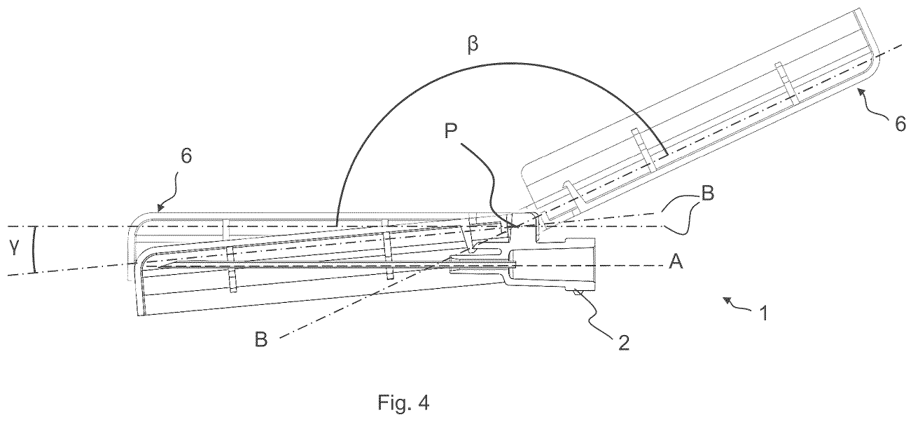

When pivoting the sheath 6 between the starting position and the use position, an angular displacement beta is done of more than 90 degrees, as shown in FIG. 4. The angular displacement beta is the angular displacement of the longitudinal axis B of the sheath 6 between the starting position and the use position. Preferably, the angular displacement beta is more than 90 degrees, more preferably more than 100 degrees, and more preferably more than 110 degrees.

The angular displacement of the sheath 6 between the starting position and the locked position is indicated by gamma. The angular displacement gamma, shown in FIG. 4, is advantageously less than 20 degrees, preferably less than 15 degrees and more preferably less than 8 degrees. The angular displacement gamma is obtained as the angular displacement of the longitudinal axis B of the sheath 6 between the starting position and the locked position. The longitudinal axis B of the sheath 6 is advantageously arranged through a pivot axis P of the pivot connection 7.

Also, as can be seen in FIGS. 3a, 3b and 3c, the hub 2 and the sheath 6 are provided with cooperating elements that cooperate with each other in the starting position and in the locked position. The hub 2 is provided with at least one first cooperating element 23 and the sheath 6 is provided with at least one second cooperating element 24. The at least one first cooperating element 23 and the at least one second cooperating element 24 are associated to each other and cooperate with each other in the starting position and in the locked position. In the starting position, the first cooperating element 23 and the second cooperating element 24 engage to each other to define the starting position. In the locked position, the first cooperating element 23 and the second cooperating element 24 cooperate with each other to lock the sheath.

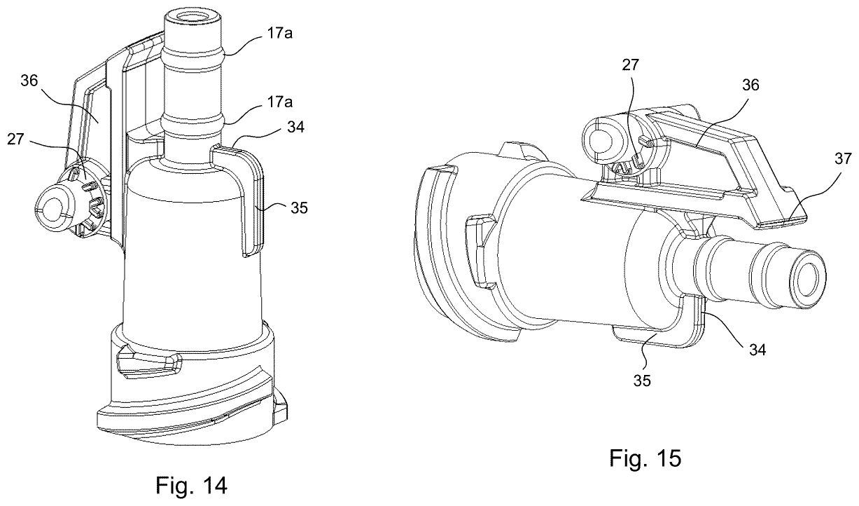

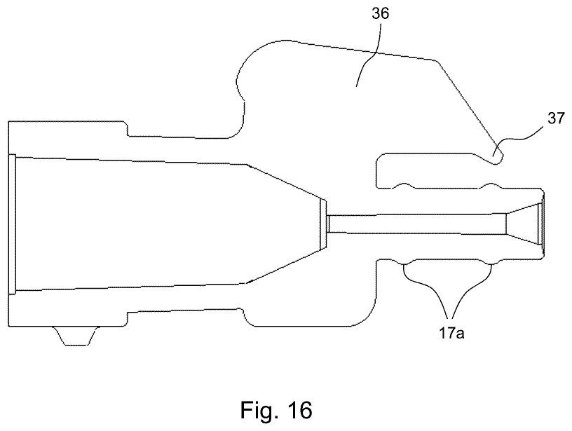

The hub 2 may further comprise an extension part 36 extending from the hub 2 at least partially in a direction towards the sheath 6 and approximately parallel to the protrusion 17, at least partially arranged between the sheath 6 and the protrusion 17. The extension part 36 may comprise a tomahawk-like shaped reinforcing part, see FIG. 14-16. The shape may be designed with use of finite element analysis taking into account the forces exerted thereon. The extension part 36 may comprise at least one further first cooperating element 37 that, in the locked position, cooperates with at least one corresponding further second cooperating element arranged on the sheath 6. The at least one further first cooperating element 37 may be arranged as a protruding edge extending from the extension part 36, e.g. arranged in a barb shape for absorbing large forces, and the corresponding at least one further second cooperating element may be arranged as a hook extending from the sheath 6. The barb-shaped edge of the extension part 36 of the hub 2 and the hook of the sheath 6 engage when the locked position is activated such that the hook is snapped around the edge.

In the embodiment of FIGS. 3a, 3b, 3c, the first cooperating element 23 is embodied as a protruding finger, longitudinally protruding from the distal end 4 of the hub 2. The second cooperating element 24 is, in the embodiment of FIGS. 3a, 3b, 3c, provided as a hook 24 extending downwardly from the back wall 14 inside of the receiving space 15. In the locked position, the hook 24 hooks behind the finger 23 to lock the sheath and to secure the needle 5 in the receiving space 15 of the sheath 6. Here, in the starting position, the hook 24 rests onto the finger 23. The hook 24 and the finger 23 have an inclined or chamfered contact surface 24a, 23a at their respective ends that can contact each other. The inclination of the chamfered surfaces 23a, 24a is the same such that the surfaces 23a, 24a can optimally contact each other. Advantageously, the angle of inclination of the surfaces 23a, 24a is 45 degrees. The hook 24 further is provided with a further contact surface 24b at its end, the contacting surfaces 24a and 24b together defining a hook shape. In the locked position, the contact surface 24b hooks behind the finger 23 to contact an underside 23b of the finger 23 and thus to lock. The first and second cooperating elements 23, 24 engage in a snap-fit connection in the locked position by the second cooperating element 24 snap-fitting behind the first cooperating element 23.

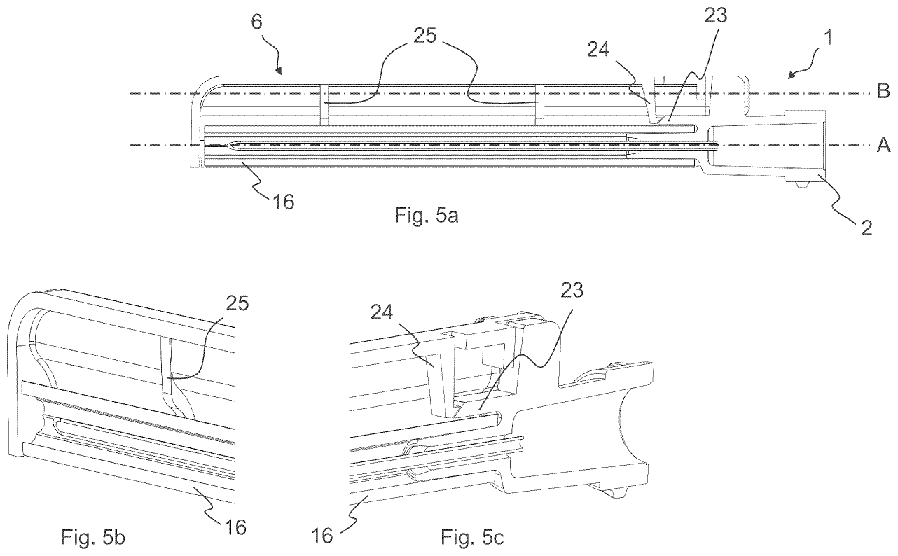

FIGS. 5a, 5b, 5c show an example of a safety needle assembly 1 with the sheath 6 in the starting position. The first and second cooperating elements 23, 24 are here embodied as a protruding finger 23 and a downwardly extending hook 24, similar as in the embodiment of FIGS. 3a-3c. The safety needle assembly 1 further comprises a protector 16. In FIG. 5a, and in the detail thereof in FIG. 5b, it can be seen that the sheath 6 further comprises bridge elements 25 that are arranged in the receiving space 15 of the sheath 6. The bridge elements 25 support on the protector 16 in the starting position of the sheath 6, as can be seen in FIG. 5a and FIG. 5b. The bridge elements 25 may be shaped to fit an outer surface of the protector 16. By providing the bridge elements 25 supporting on the protector 16 in the starting position, these bridge elements 25 provide for a stop of the sheath in the starting position. The starting position of the sheath 6 is then not only defined by the first and second cooperating elements 23, 24 abutting each other, but also by the bridge elements 25 supporting on the protector. The bridge elements 25 may prevent accidental activation of the sheath 6, for example may prevent that the sheath 6 moves out of the starting position, e.g. accidentally by forces during transport and/or handling. By providing the protector 16, an accidental needle stick may be prevented when opening the sheath from the starting position towards the use position. Also, by combination of the bridge elements 25 supporting on the protector 16 de-activation of the sheath out of the starting position during transport and manipulation can be obviated. Bridge elements 25 close to the hub 2 may also prevent relative movement during connection of the syringe to the hub 2. Multiple bridge elements 25 can be provided distributed over the length of the sheath 6 in the receiving space 15 of the sheath 6.

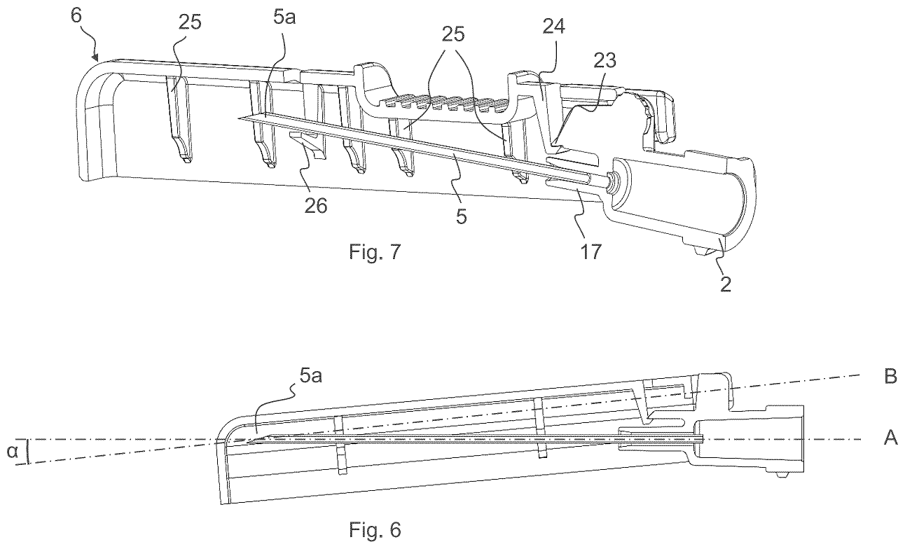

FIG. 6 shows the sheath 6 in the locked position, wherein the angle alpha is determined by the longest needle possible in the safety needle assembly in combination with the available sheath 6. The sheath 6 cannot pivot further downward, with the longest needle connected to the hub, as, in the locked position, the distal end 5a of the needle 5 abuts the sheath 6. As such, the angular displacement of the sheath 6 is limited. Typically, the angle alpha in the locked position is about minus 10 degrees, preferably about minus 8 degrees, more preferably about minus 6 degrees.

By providing the starting position of the sheath 6 in a longitudinally extending position from the hub 2, and with a limited angular displacement between the starting position and the locked position of the sheath, the first and second cooperating elements 23, 24 are advantageously robust and provide for a reliable locking. By providing a snap-fit engagement as with the snap hook 24 and the snap finger 23, such a robust and reliable connection can be obtained. Due to the small angular displacement of the sheath 6 between the starting position and the locked position, the actual displacement of the hook 24 with respect to the finger 23 is relatively small, for an angle of about 6 degrees this is about a 1 mm displacement. Taking into a general material thickness of such injection molded objects of about 0.8 mm, a displacement of about 1 mm, is just sufficient to overcome the thickness of the snap hook 24. Also, for providing a robust snap fit connection, it is advantageous to have the snap finger 23 as short as possible, however, given the small angular displacement and the minimum height displacement to overcome the material thickness, also the snap finger 23 needs to be given some length to protrude out of the hub 2. When the snap finger 23 is as short as possible for robust snapping, the snap finger is more rigid, less material elasticity is possible in such a short finger. Also, after locking, the connection between the first cooperating element 23 and the second cooperating element 24 needs to be reliable, it may not be come loose accidentally due to e.g. handling forces. But to provide for a reliable connection in the locked position, it is beneficial to have the snap fit connection as far away from the pivot axis as possible. This requirement is contradictory to at least the requirement for a robust snapping for which a short finger is preferable. Combining these contradictory requirements provides for first and second cooperating elements that engage in a snap fit connection for locking the sheath and that abut to each other in the starting position for defining the starting position of the sheath.

In FIG. 7 can be seen that the sheath 6 further is provided with an additional cooperating element 26 for engaging with a distal end 5a of the needle 5 in the locked position of the sheath 6. By providing such an additional cooperating element 26, here embodied as a seat on which the distal end 5a rests in locked position, an additional support to the needle 5 is given in the locked position, thereby obviating accidental unlocking of the needle, e.g. due to bending of the needle in view of the relative elasticity of a rather long needle. The seat 26 is at an under side provided with a chamfered or inclined surface, such that the needle end 5a can be easily guided along it during the locking movement, but while the seat may prevent the needle end 5a moving downward again, or, vice versa, may prevent the sheath from moving out of the locked position.

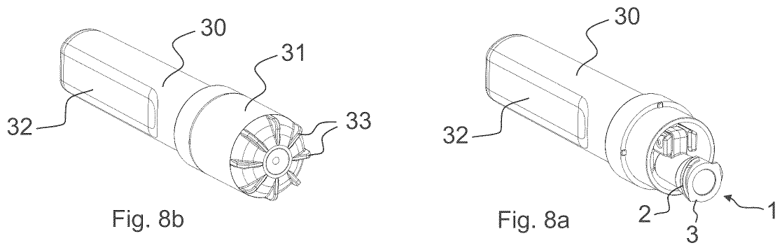

FIGS. 8a and 8b show the assembly 1 in the starting position, in which it can be packaged by a hard case 30 with a hard cap 31. Due to the compact volume of the assembly 1 in the starting position, due to the longitudinally extending position of the sheath 6 in the starting position, the assembly 1 may fit in a relatively small volume hard case. Contrary to the conventional packaging, typically blister packaging, the hard case can be handled automated during manufacturing and assembly, as well as provides for more space efficiency during transport and/or handling. The hard case 30 can have a cylindrical body 32. In the embodiment shown in FIGS. 8a, 8b, the body is of a more rectangular shape. Such a shape may increase the grip for the user during handling of the hard case 30, as well as may prevent rolling over when lying the hard case 30 on a surface, e.g. prior to the preparation of the injection needle. Further, the rectangular shape may facilitate automated handling, as it may be more easily gripped by a robot.

The assembly 1 fits inside of the hard case 30, that is provided with holding elements inside thereof, e.g. to prevent accidental fall out of the assembly 1 when the hard case is held upside down. The hub 2 may be provided with a means for connecting with the case 30, e.g. a rib 35 as illustrated in FIG. 14, extending from an outer surface of the hub 2, radially away from the hub 2, wherein the rib may fit into a corresponding groove arranged on an inner surface of the case 30. This is especially advantageous in order to prevent relative movement of the hub 2 with respect to the case 30 during mounting of the syringe onto the hub 2. Further, the sheath 6 may be provided with a means for connecting with the case 30, e.g. dents or grooves, on an outer surface, e.g. on a distal end, of the sheath 6. On an inner surface of the case 30, corresponding connection means may be arranged. By providing such additional connections, the needle assembly 1 is further stabilized within the case 30 and sterility of the product is further ensured. The cap 31 can be fitted onto the case 30 e.g. by clicking, screwing, clamping etc. The assembly 1 is packaged sterile in the case 30 and cap 31. Prior to the use of the assembly 1, the user removes the cap 31 from the case 30. The ribs 33 on the cap 31 may facilitate removal. With the assembly 1 remaining in the case 30, the syringe is connected to the proximal end 3 of the hub 2. When the syringe is then connected to the hub 2, the assembly 1 is removed from the case 30. Then, the sheath 6 can be pivoted from the starting position towards the use position for exposing the needle 5. And, after injection, the sheath 6 can be pivoted towards the locked position for locking the sheath. By providing such a hard case, that can hold the assembly 1, handling of the assembly can be done relatively long with the assembly still in the case 30, thereby optimally maintaining the sterility of the assembly 1.

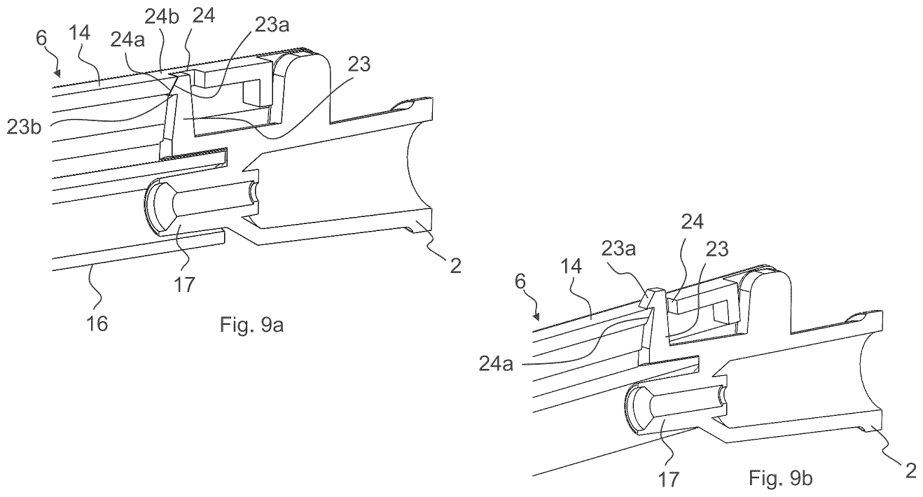

FIGS. 9a and 9b show an alternative embodiment of the first and second cooperating elements 23, 24. Here, the first cooperating element 23 is a snap hook 23 extending from the hub in an L-shape. The cooperating second element 24 is an opening 24 in the back wall 14 of the sheath 6. The opening 24 and the snap hook 23 have corresponding chamfered surfaces 23a. 24a that abut against each other in the starting position of the sheath 6 to define the starting position of the sheath 6. In the locked position, the snap hook 23 is snapped through the opening 24 to clamp behind the back wall 14. A surface 23b of the snap hook 23 engages with the outer surface of the back wall 14 in the locked position to provide for a firm, robust and reliable snap fit connection. Here, the surfaces 23b and 23a may provide for the hook shape of the hook 23. The inclined contact surfaces 23a, 24a contact each other in the starting position of the sheath, to define the starting position. Whereas the further contact surfaces 23b, 24b engage each other in the locked position. The configuration of contact surfaces 23a, 24a and further contact surfaces 23b, 24b provides for the snap fit connection in the locked position of the sheath.

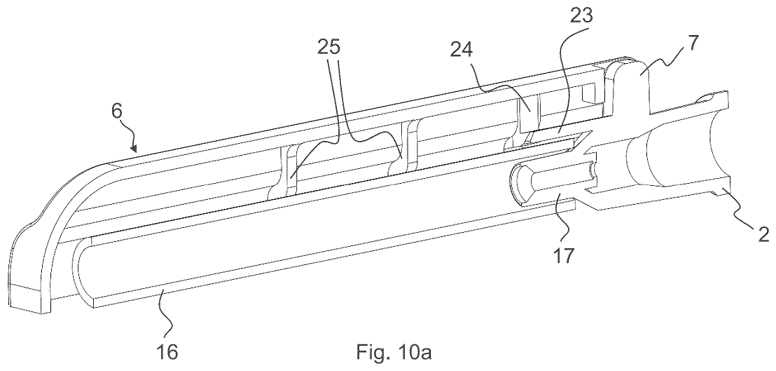

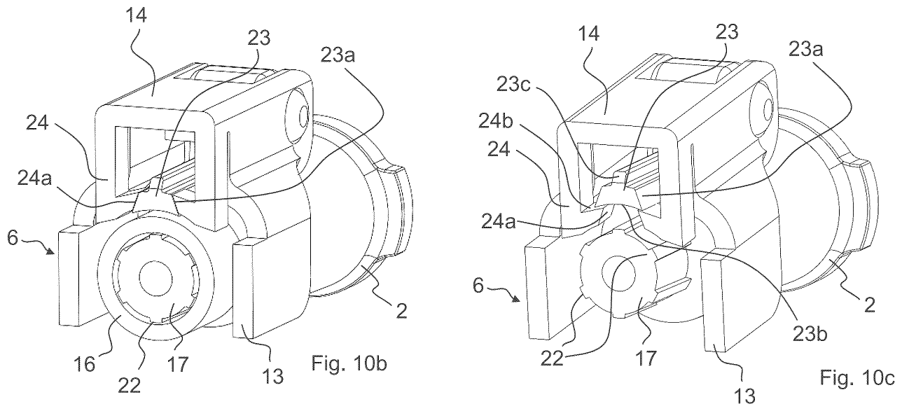

FIGS. 10a, 10b and 10c show an alternative embodiment of the first cooperating elements 23 and the second cooperating elements 24. In FIG. 10a, a perspective view is shown of the sheath 6 in the starting position. The sheath 6 having bridge elements 25 supporting on the protector 16. The first and second cooperating elements 23, 24 are shown in detail in FIG. 10b, in the starting position, and FIG. 10c, in the locked position. The first cooperating element 23 is here embodied as a finger 23 longitudinally extending from the hub 2. The second cooperating elements 24 are embodied as two hook shaped elements 24 extending downwardly from the back wall 14 of the sheath 6, each at a side wall 13 of the sheath 6. The hooks 24 are connected at one end only to the back wall 14 or the side wall 13, but are free otherwise from the sheath 6, thus providing for some resiliency. The hooks 24 may thus be flexible or movable with respect to their connected end. In the starting position, as can be seen in FIG. 10b, the hooks 24 rest onto the finger 23. Surfaces 24a of the hooks 24 and surfaces 23a of the finger 23 contact each other. The hooks 24 have chamfered or inclined surfaces 24a that are configured for abutting onto corresponding chamfered surfaces 23a of the finger 23. The finger 23 is provided with two chamfered surfaces 23a, one surface 23a at each side of the finger 23, such that a corresponding associated surface 24a can contact the respective surface 23a. For an advantageous contact in the starting position of the sheath 6, the chamfer or inclination of the surfaces 23a, 24a is preferably the same or similar. The angle of inclination of the surfaces 23a, 24a is advantageously such that in the starting position, the surfaces 23a, 24a can easily rest onto each other, while, when additional force is applied to push the sheath 6 towards the locked position, that the surfaces 23a, 24a can then slide along each other towards the locked position. In the locked position, another surface 24b of the hook 24 contacts an underside 23b of the finger 23 to form a secure snap-fit connection. In this embodiment, the finger 23 is provided with an additional rib 23c, longitudinally extending on an upper side of the finger 23, to provide additional stiffness to the finger 23.

In FIGS. 10b, 10c, it can be seen that the protrusion 17 in which the needle 5 is received is provided with ribs 22 to which the protector 16 can clampingly engage. Providing ribs 22 onto the protrusion 17 may be considered as an alternative to the longitudinal ribs 16a at an inner side of the protector 16 as shown in FIG. 1. In this example, a protector 16 having a smooth inner surface may be considered to use.

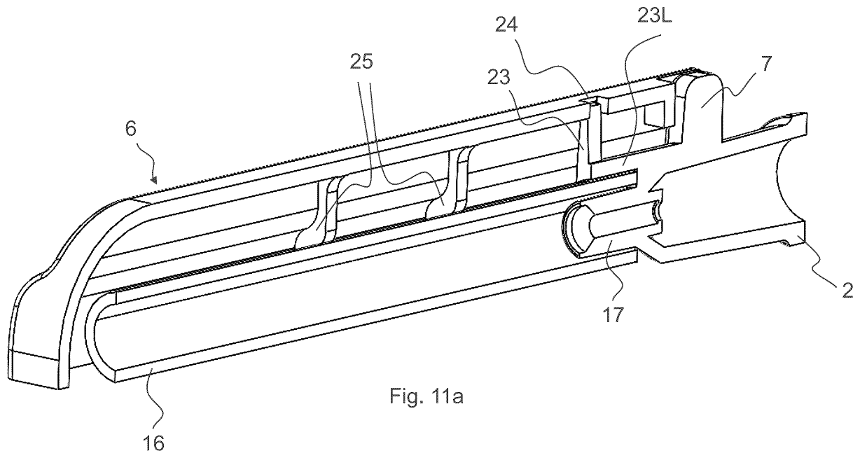

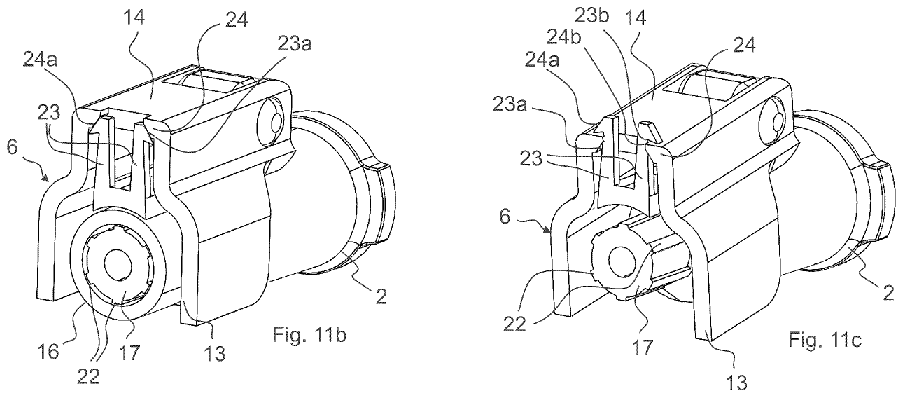

The embodiment of FIGS. 11a, 11b, 11c show an alternative embodiment of the first cooperating elements 23, and the second cooperating elements 24. In the perspective view of FIG. 11a can be seen that, here too, the sheath 6 is provided with bridge elements 25 that support onto the protector 16 in the starting position. In the starting position, the sheath 6 is longitudinally extending from the hub, in the same direction as the needle 5 and the protector 16. FIG. 11b shows a detail of the first and second cooperating elements 23, 24 in the starting position. FIG. 11c shows a detail of the first and the second cooperating elements 23, 24 in the locked position. Here, the first cooperating elements 23 are embodied as two hooks extending in an L-shape from the hub 2. The hooks 23 have a common base, finger 231, that longitudinally extends from the hub 2, from which base 231 the hooks 23 protrude upwardly. The hooks 23 are provided with inclined contact surfaces 23a that abut onto the associated inclined contact surfaces 24a of the second cooperating elements 24, as can be seen in FIG. 11b. The second cooperating element 24 is here embodied as an opening in the back wall 14 of the sheath 6. Side surfaces 24a of the opening 24 are inclined such that they can contact the inclined surfaces 23a of the hooks 23 in the starting position. When the sheath 6 is pushed further through towards the locked position, further contact surfaces 23b provided at the hook 23 engage the upper surface of the back wall 14, as can be seen in FIG. 11c. The upper surface of the back wall 14 thus serving as the contact surface 24b for engaging with the surface 23b of the second cooperating element 23 in the locked position.

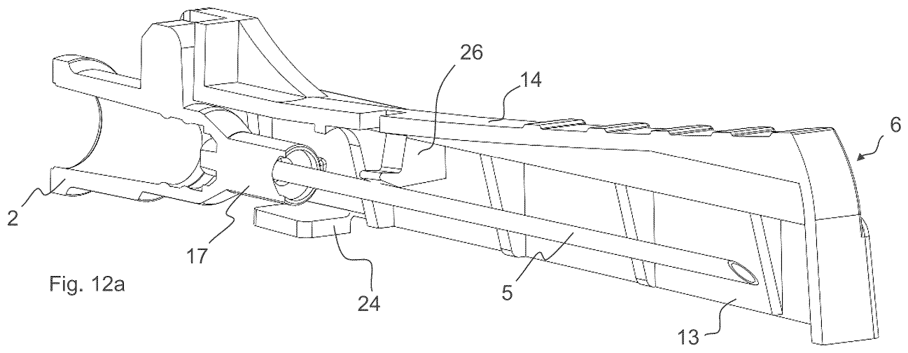

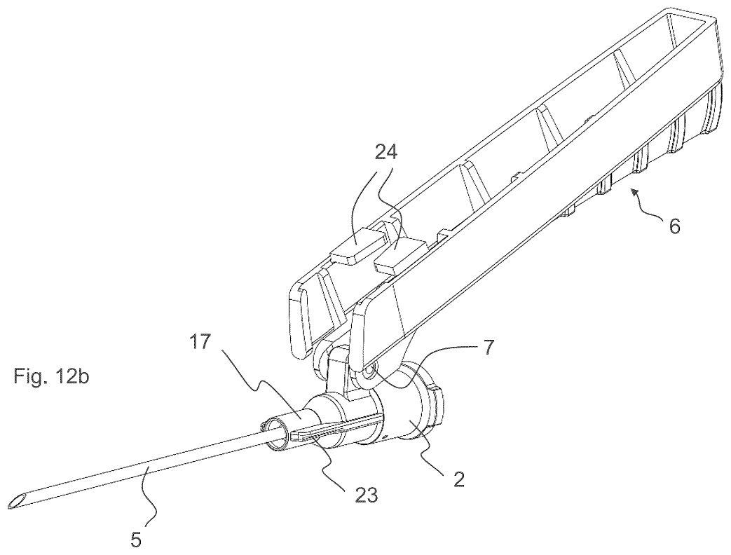

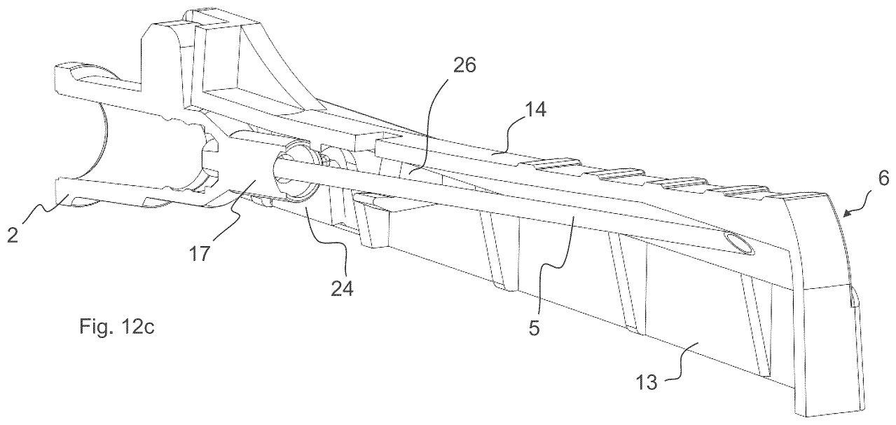

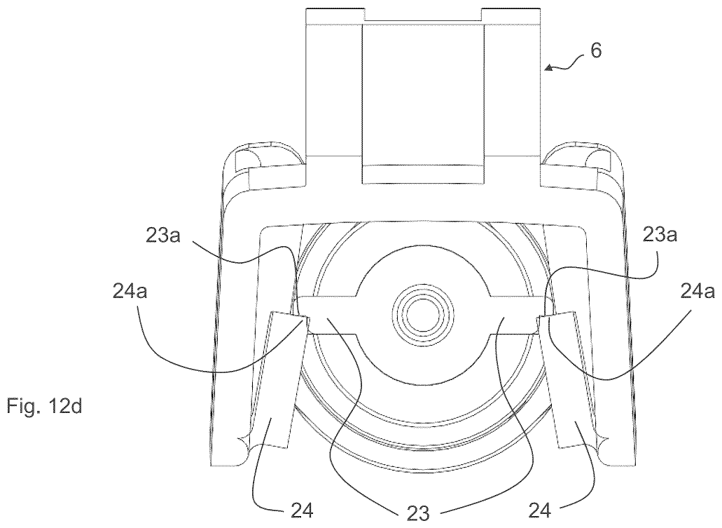

FIGS. 12a-12d show another embodiment of the first cooperating elements 23 and the second cooperating elements 24. Here, the first cooperating elements 23 are embodied as ribs 23 onto the protrusion 17 in which the needle 5 can be connected. The second cooperating elements 24 are here embodied as wings 24 pivotable connected to the side walls 13 of the sheath 6. The wings 24 may be connected to the side walls 13 via a living hinge. In the starting position, shown in FIG. 12a, the wings 24 are oriented towards each other such that the wings 24 face each other. The wings 24 are thus oriented in a direction transverse to the side walls 13 of the sheath 6, as to close off the receiving space 15 with the wings 24. Advantageously, the wings 24 are biased towards this closed position. Thus, in the starting position, the sheath 6 encloses the needle 5 since the wings 24 are closed below the needle 5. When pivoting the sheath 6 from the starting position towards the use position, the wings 24 pass along the protrusion 17 and are being forced outwardly when passing the protrusion 17, but after passing the protrusion 17 the wings 24 pivot back towards the closed position to which they are biased. The use position of the sheath 6 is shown in FIG. 12b. After use of the injection needle 5, the safety sheath 6 is pivoted towards the locked position to secure the needle 5 in the receiving space 15 of the sheath 6. When pivoting the sheath 6 towards the locked position, the wings 24 pass along the protrusion 17 with the ribs 23 again, now being forced in an inward position pivoted toward the side walls 13 of the sheath 6, as can be seen in FIG. 12c. When pushing the sheath 6 further down towards the locked position, the wings 24 at a certain moment engage with ends of the ribs 23. The ribs 23 are provided with end surfaces 23a, as a cut out in the rib 23, in which cut out 23a an edge surface 24a of the wings 24 may engage. Due to the bias of the wings 24 towards the closed position, the wings 24 clampingly engage the cut out surfaces 23a and a firm locking of the wings 24 to the ribs 23 is obtained, as can be seen in FIG. 12d.

It will be appreciated that many variants of the first and second cooperating elements are possible. Some of those variants are described above.

For the purpose of clarity and a concise description, features are described herein as part of the same or separate embodiments, however, it will be appreciated that the scope of the claims and disclosure may include embodiments having combinations of all or some of the features described. It may be understood that the embodiments shown have the same or similar components, apart from where they are described as being different.

In the claims, any reference signs placed between parentheses shall not be construed as limiting the claim. The word ‘comprising’ does not exclude the presence of other features or steps than those listed in a claim. Furthermore, the words ‘a’ and ‘an’ shall not be construed as limited to ‘only one’, but instead are used to mean ‘at least one’, and do not exclude a plurality. The mere fact that certain measures are recited in mutually different claims does not indicate that a combination of these measures cannot be used to an advantage. Many variants will be apparent to the person skilled in the art. All variants are understood to be comprised within the scope defined in the following claims.

1. A safety needle assembly comprising:
a hub having a proximal end for connecting with a syringe extending in a proximal area of the hub;
a needle longitudinally extending from a distal end of the hub in a distal area of the hub;
a sheath pivotally connected to the hub,
the sheath having two side walls connected by a back wall defining a receiving space therebetween for receiving the needle;
wherein the sheath is adjustable between a starting position in which the sheath is substantially positioned in the distal area of the hub, between a use position in which the sheath is substantially positioned in the proximal area of the hub, and between a locked position in which the sheath is locked to secure the needle into the receiving space;
wherein in the starting position the sheath is longitudinally extending from the hub, wherein the hub is provided with at least one first cooperating element and the sheath is provided with at least one second cooperating element, wherein, in the locked position of the sheath, the first and the second cooperating elements are engaged to each other for locking the sheath, wherein, in the starting position, the first and second cooperating elements are in contact with each other for defining the starting position of the sheath.

2. A safety needle assembly comprising:
a hub having a proximal end for connecting with a syringe extending in a proximal area of the hub;
a needle longitudinally extending from a distal end of the hub in a distal area of the hub;
a sheath pivotally connected to the hub,
the sheath having two side walls connected by a back wall defining a receiving space therebetween for receiving the needle;
wherein the sheath is adjustable between a starting position in which the sheath is substantially positioned in the distal area of the hub, between a use position in which the sheath is substantially positioned in the proximal area of the hub, and between a locked position in which the sheath is locked to secure the needle into the receiving space;
wherein in the starting position the sheath is longitudinally extending from the hub, wherein the hub is provided with at least one first cooperating element and the sheath is provided with at least one second cooperating element, wherein, in the locked position of the sheath, the first and the second cooperating elements are engaged to each other for locking the sheath, wherein, the first cooperating element protrudes in a direction away from the hub parallel to the cannula extending from the hub and the second cooperating element protrudes from the back wall of the sheath in a direction approximately parallel to the side walls into the receiving space.

3. The safety needle assembly according to claim 1, wherein the at least one first cooperating element longitudinally extends from the distal end of the hub.

4. The safety needle assembly according to claim 2, wherein the first and second cooperating elements engage with each other in the starting position, defining the starting position by abutting to each other.

5. The safety needle assembly according to claim 1, wherein the at least one second cooperating element protrudes from the back wall of the sheath into the receiving space, and/or, wherein the at least one second cooperating element is at least one opening in the sheath.

6. The safety needle assembly according to claim 1, wherein at least one of the first and second cooperating elements is provided as a snap fit element that engages in a snap-fit connection in the locked position of the sheath.

7. The safety needle assembly according to claim 1, wherein the first and second cooperating elements each are provided with inclined contact surfaces that are configured for contacting each other in the starting position of the sheath, the contact surfaces having the same inclination angle.

8. The safety needle assembly according to claim 7, wherein the first and second cooperating elements further are provided with further contact surfaces, at a different angle than the inclined contact surfaces, wherein the further contact surfaces are configured for contacting each other in the locked position of the sheath.

9. The safety needle assembly according to claim 1, wherein the angular displacement of the sheath between the starting position and the locked position of the sheath is less than 20 degrees.

10. The safety needle assembly according to any claim 1, wherein the angular displacement of the sheath between the starting position and the use position is more than 90 degrees.

11. The safety needle assembly according to claim 1, further comprising a protector removable engageable to the distal end of the hub for protecting the needle when the sheath is in the starting position.

12. The safety needle assembly according to claim 1, wherein the sheath comprises bridge elements arranged in the receiving space which bridge elements support on the protector in the starting position of the sheath.

13. The safety needle assembly according to claim 1, wherein the sheath further is provided with an additional cooperating element for engaging with a distal end of the needle in the locked position of the sheath.

14. The safety needle assembly according to claim 1, wherein the sheath comprises a recess region to engage with a finger of a user, such that the user can pivot the sheath between the starting position, the use position and/or the locked position.

15. A set of a safety needle assembly according to claim 1 and a hard case comprising a cap and a case in which case the safety needle assembly is inserted when the sheath is in the starting position.

16. A hard case packaging comprising a case in which the safety needle assembly according to claim 1 is insertable, wherein the hard case packaging is closable by a hard cap.

17. A kit of the safety needle assembly according to claim 1, and the hard case packaging according to claim 16.
